(19) United States
(12) Patent Application Publication
(10) Pub. No.: US 2017/0030854 A1
(43) Pub. Date: Feb. 2, 2017

(54) SENSOR FOR CHEMICAL ANALYSIS AND METHODS FOR MANUFACTURING THE SAME

(71) Applicant: LIFE TECHNOLOGIES CORPORATION, Carlsbad, CA (US)

(72) Inventors: Creighton BUIE, Daly City, CA (US); John DONOHUE, Southbury, CT (US); Chiu Tai Andrew WONG, Orange, CT (US); James A. BALL, Ledyard, CT (US); Wolfgang HINZ, Killingworth, CT (US); James BUSTILLO, Castro Valley, CA (US); Jordan OWENS, Austin, TX (US); Shifeng LI, Fremont, CA (US)

(21) Appl. No.: 15/222,492

(22) Filed: Jul. 28, 2016

Related U.S. Application Data
(60) Provisional application No. 62/198,967, filed on Jul. 30, 2015.

(51) Int. Cl. G01N 27/327; C12Q 1/68

(52) U.S. Cl. CPC G01N 27/3275; C12Q 1/6874

(57) ABSTRACT

Provided herein is a sensor comprising a substrate having a first reaction region and a second reaction region, a first electrode associated with the first reaction region, a second electrode associated with the second reaction region and a third electrode wherein the third electrode is common to both the first reaction region and the second reaction region.

### CROSS-REFERENCE

This application claims priority to U.S. Provisional Application No. 62/198,967, filed on Jul. 30, 2015.

### BACKGROUND

The present disclosure relates to sensors for chemical analysis, and to methods for manufacturing such sensors.

A variety of types of sensors have been used in the detection of chemical processes. One type is a chemically-sensitive field effect transistor (chemFET). A chemFET includes a source and a drain separated by a channel region, and a chemically sensitive area coupled to the channel region. The operation of the chemFET is based on the modulation of channel conductance, caused by changes in charge at the sensitive area due to a chemical reaction occurring nearby. The modulation of the channel conductance changes the threshold voltage of the chemFET, which can be measured to detect and/or determine characteristics of the chemical reaction. The threshold voltage may for example be measured by applying appropriate bias voltages to the source and drain, and measuring a resulting current flowing through the chemFET. As another example, the threshold voltage may be measured by driving a known current through the chemFET, and measuring a resulting voltage at the source or drain.

An ion-sensitive field effect transistor (ISFET) is a type of chemFET that includes an ion-sensitive layer at the sensitive area. The presence of ions in an analyte solution alters the surface potential at the interface between the ion-sensitive layer and the analyte solution, due to the protonation or deprotonation of surface charge groups caused by the ions present in the analyte solution. The change in surface potential at the sensitive area of the ISFET affects the threshold voltage of the device, which can be measured to indicate the presence and/or concentration of ions within the solution.

Arrays of ISFETs may be used for monitoring chemical reactions, such as DNA sequencing reactions, based on the detection of ions present, generated, or used during the reactions. More generally, large arrays of chemFETs or other types of sensors may be employed to detect and measure static and/or dynamic amounts or concentrations of a variety of analytes (e.g. hydrogen ions, other ions, compounds, etc.) in a variety of processes. The processes may for example be biological or chemical reactions, cell or tissue cultures or monitoring neural activity, nucleic acid sequencing, etc.

An issue that arises in the operation of large scale sensor arrays is the susceptibility of the sensor output signals to noise. For example, the noise affects the accuracy of the downstream signal processing used to determine the characteristics of the chemical and/or biological process being detected by the sensors. Also, byproducts of the chemical and/or biological process being detected are produced in small amounts or rapidly decay or react with other constituents.

It is therefore desirable to provide devices including low noise sensors, sensors providing novel means for detection, and methods for manufacturing such devices.

### BRIEF DESCRIPTION OF THE DRAWINGS

The accompanying drawings, which are incorporated herein and form a part of the specification, illustrate embodiments of the present invention and, together with the description, further serve to explain the principles of the invention and to enable a person skilled in the relevant art to make and use the invention.

FIG. 1 illustrates a block diagram of components of a chemical/biological detection system according to an exemplary embodiment.

FIG. 2 illustrates a cross-sectional view of a portion of the integrated circuit device and flow cell according to an exemplary embodiment, and an expanded view of a sensor and corresponding reaction region.

FIG. 3 illustrates a cross-sectional of representative sensors and corresponding reaction regions according to an exemplary embodiment.

FIGS. 4 to 11 illustrate stages in a manufacturing process for forming an array of sensors and corresponding well structures according to an exemplary embodiment.

FIG. 12 illustrates an exemplary routing scheme of the integrated circuit

FIG. 13 illustrates an exemplary flow chart according to an exemplary embodiment.

### DETAILED DESCRIPTION

The following detailed description refers to the accompanying drawings that illustrate exemplary embodiments consistent with this invention. Other embodiments are possible, and modifications can be made to the embodiments within the scope of the invention. Therefore, the detailed description is not meant to limit the invention.

It would be apparent to person of ordinary skill in the relevant art that the present invention, as described below, can be implemented in many different embodiments of hardware and/or the entities illustrated in the figures. Thus, the operational behavior of embodiments of the present invention will be described with the understanding that modifications and variations of the embodiments are possible, given the level of detail presented herein.

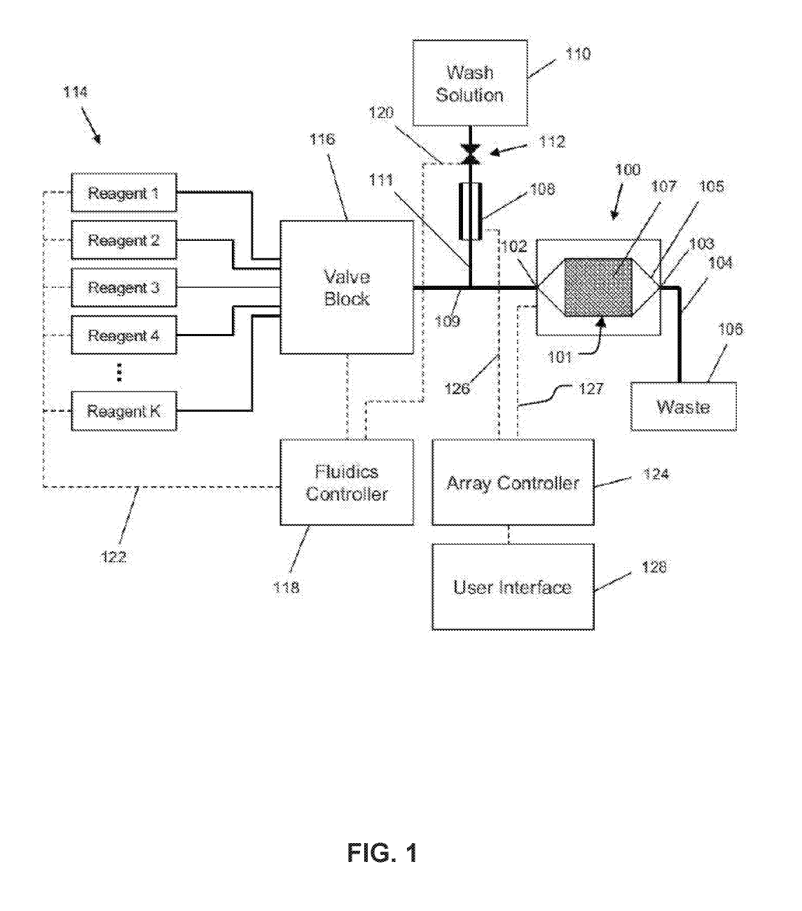

FIG. 1 illustrates a block diagram of components of a system for nucleic acid sequencing according to an exemplary embodiment. The components include a flow cell 101 on an integrated circuit device 100, a reference electrode 108, a plurality of reagents 114 for sequencing, a valve block 116, a wash solution 110, a valve 112, a fluidics controller 118, lines 120/122/126, passages 104/109/111, a waste container 106, an array controller 124, and a user interface 128. The integrated circuit device 100 includes a microwell array 107 overlying a sensor array that includes sensors as described herein. The flow cell 101 includes an inlet 102, an outlet 103, and a flow chamber 105 defining a flow path of reagents over the microwell array 107. The reference electrode 108 can be of any suitable type or shape, including a concentric cylinder with a fluid passage or a wire inserted into a lumen of passage 111. The reagents 114 can be driven through the fluid pathways, valves, and flow cell 101 by pumps, gas pressure, or other suitable methods, and can be discarded into the waste container 106 after exiting the outlet 103 of the flow cell 101. The fluidics controller 118 can control driving forces for the reagents 114 and the operation of valve 112 and valve block 116 with suitable software.

The microwell array 107 includes an array of reaction regions as described herein, also referred to herein as microwells, which are operationally associated with corresponding sensors in the sensor array. For example, each reaction region may be coupled to a sensor suitable for detecting an analyte or reaction property of interest within that reaction region. The microwell array 107 may be integrated in the integrated circuit device 100, so that the microwell array 107 and the sensor array are part of a single device or chip. The flow cell 101 can have a variety of configurations for controlling the path and flow rate of reagents 114 over the microwell array 107. The array controller 124 provides bias voltages and timing and control signals to the integrated circuit device 100 for reading the sensors of the sensor array. The array controller 124 also provides a reference bias voltage to the reference electrode 108 to bias the reagents 114 flowing over the microwell array 107. During an experiment, the array controller 124 collects and processes output signals from the sensors of the sensor array through output ports on the integrated circuit device 100 via bus 127. The array controller 124 may be a computer or other computing means. The array controller 124 may include memory for storage of data and software applications, a processor for accessing data and executing applications, and components that facilitate communication with the various components of the system in FIG. 1.

The values of the output signals of the sensors indicate physical and/or chemical parameters of one or more reactions taking place in the corresponding reaction regions in the microwell array 107. The user interface 128 may display information about the flow cell 101 and the output signals received from sensors in the sensor array on the integrated circuit device 100. The user interface 128 may also display instrument settings and controls, and allow a user to enter or set instrument settings and controls. In some embodiments, during the experiment the fluidics controller 118 may control delivery of the individual reagents 114 to the flow cell 101 and integrated circuit device 100 in a predetermined sequence, for predetermined durations, at predetermined flow rates. The array controller 124 may then collect and analyze the output signals of the sensors indicating chemical reactions occurring in response to the delivery of the reagents 114. During operation, the system may also monitor and control the temperature of the integrated circuit device 100, so that reactions take place and measurements are made at a known predetermined temperature. The system may be configured to let a single fluid or reagent contact the reference electrode 108 throughout an entire multi-step reaction during operation. The valve 112 may be shut to prevent any wash solution 110 from flowing into passage 109 as the reagents 114 are flowing. Although the flow of wash solution can be stopped, there may be uninterrupted fluid and electrical communication between the reference electrode 108, passage 109, and the microwell array 107. The distance between the reference electrode 108 and the junction between passages 109 and 111 may be selected so that little or no amount of the reagents flowing in passage 109 and possibly diffusing into passage 111 reach the reference electrode 108. In some embodiments, the wash solution 110 may be selected as being in continuous contact with the reference electrode 108, which can be especially useful for multi-step reactions using frequent wash steps.

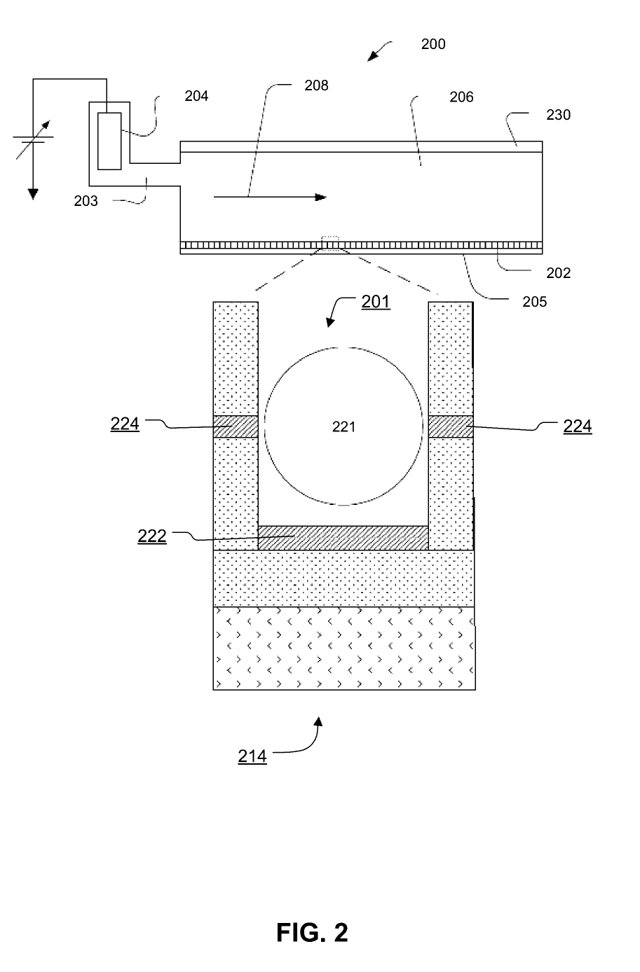

FIG. 2 is an expanded and cross-sectional view of a flow cell 200 and shows a portion of a flow chamber 206. A reagent flow 208 flows across a surface of a microwell array 202, in which the reagent flow 208 flows over the open ends of the microwells. The microwell array 202 and a sensor array 205 together can form an integrated unit forming a bottom wall (or floor) of flow cell 200. A reference electrode 204 can be fluidly coupled to flow chamber 206. Further, a flow cell cover 230 may serve as the top surface of the flow chamber 206 and may provide a volume for reagent flow 208 in the flow cell.

A detailed view of an exemplary microwell 201 and a sensor 214 is also illustrated in FIG. 2. The sensor can include electrode 222 at the bottom of microwell 201 and common electrode or reference electrode 224 disposed within sidewalls of the dielectric layer forming the well wall. The common electrode may be located at any suitable position within the dielectric layer. In some embodiments, the common electrode is positioned such that it maximized the amount of nucleic acids, such as for example, DNA, between the common electrode and the sensor. In an embodiment, reactions carried out in microwell 201 can be analytical or biological reactions to detect or identify characteristics or properties of an analyte of interest. In some embodiments, a sensor may have two parallel plates including two electrodes. An analyte (e.g. DNA) is loaded between the two electrodes, whereby a modulation of conductance through the DNA can be measured. The analyte may serve as the basis for or contribute to the charge in the solution between the plates (counter ions being the charge carriers). For example, an analyte physically present between the two electrodes may serve as the basis for or contribute to the signal. In some embodiments, an analyte may be supported by a solid support prior to deposition in a microwell. In some embodiments only a single copy of an analyte may be present. Alternatively, multiple copies of an analyte may be attached to a solid phase support 221. Only one type of analyte may be attached to the solid support (monoclonal) or multiple sample types may be attached to the solid support (polyclonal). In some embodiments, the solid phase support may be a particle, microparticle, nanoparticle, or a bead. In some embodiments the solid support may be a gel. The solid support may be porous or non-porous. Any suitable form of solid support may be used.

In some embodiments, a bead having a nucleic acid sequence may be loaded into a well, such as a microwell. The nucleic acid sequence may be DNA. The DNA may be single stranded DNA. When the bead is loaded into microwell 201, a baseline measurement may be taken such that a change in dielectric or electrical properties in a reaction region, queried area or volume (e.g. microwell 201) from an incremental change in the contents of the queried area or volume can be detected. The nucleic acid strands on the bead have an inherent charge. As a nucleotide is incorporated into the nucleic acid strands, the presence of the nucleic acid changes the charge associated with the bead via the nucleic acids. As the bead's charge increases, when immersed in a solution, the available charge within a Debye length from the chip increases, and the conductivity in this region can grow proportionally with the bead's charge, and therefore proportional to the length of the DNA extension. The bead having the nucleic acid sequence may be, for example, a porous hydrogel (e.g. similar to the current Ion Sphere Particle) or a solid particle with a hydrogel or similar coating or a solid particle with DNA directly attached to the surface). DNA can also be immobilized on a hydrogel or polymer coating located between the electrodes or on the surface of one or both of the electrodes. The number of copies of nucleic acid sequences on the solid support may be increased by any suitable amplification method including, but not limited to, rolling circle amplification (RCA), exponential RCA, RPA, emPCR, qPCR, or like techniques. Additionally, the nucleic acid may be manufactured in the microwell either with or without a solid support through any suitable manufacturing method. The volume, shape, aspect ratio (such as base width-to-well depth ratio), and other dimensional characteristics of the microwells can be selected based on the nature of the reaction taking place, as well as the reagents, byproducts, or labeling techniques (if any) that are employed.

In some embodiments, a change in dielectric or electrical property may be measured by a change in: electrical impedance; capacitance; inductance; conductance or resistance; and/or a change in resonant frequency, for example purposes only. A change in the dielectric or electrical property may be generated from an increase in molecular size or length of a nucleic acid strand present in the queried area or volume (e.g. the microwell). In some embodiments, the change may be an increase due to polymerization (including but not limited to by polymerase addition to DNA or RNA, or by protein synthesis, for example). In some embodiments, the change in the dielectric or electrical property may be a decrease in the length or molecular size of the nucleic acid or molecule(s) present in the queried area or volume. A decrease may be by attributed to either sequential or non-sequential digestion of the nucleic acid strand (including, but not limited to, exonuclease digestion of DNA or protease digestion of protein, for example). A change in the dielectric or electrical property may be generated from incorporation of additional molecules or nucleic acids to an existing nucleic acid strand or molecules present in the queried area or volume. In some embodiments, the change in the dielectric or electrical property may be generated from the binding of an antibody to an antigen. A change in the dielectric or electrical property may be generated from a disassociation of additional molecules to existing molecules in the queried are or volume. In some embodiments the disassociation may be the release of a hybridized or bound molecule from another molecule or nucleic acid strand in the queried volume.

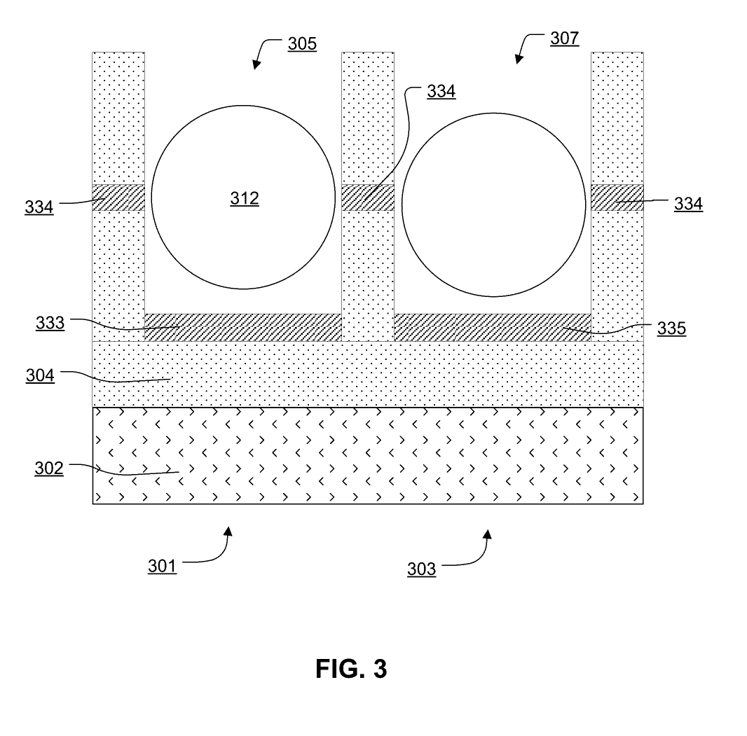

FIG. 3 illustrates adjacent sensors 301 and 303 according to an embodiment. Sensors 301, 303 each include an electrode, 333, 335, respectively, at the bottom of respective well 305, 307, and common electrode 334 is disposed within the dielectric material forming the well wall. Sensor 301 includes electrode 333. Electrode 333 can be formed on dielectric 304 which is formed on substrate 302 Dielectric 304 can comprise Tetraethyl orthosilicate, (TEOS) or silicon dioxide, for example, or any other suitable insulator material that would be known to one of skill in the art. Additional layers (for example, for signal routing) may be formed between electrode 333 and substrate 302 as has been previously described in Rotherberg et al, U.S. Pat. No. 7,948,015 and Fife, U.S. Pat. No. 8,415,176, both of which are incorporated herein by reference. Sensor 303 includes electrode 335. As with electrode 333, electrode 335 can likewise be formed on dielectric 304. The electrodes as described herein can be deposited using various techniques, such as sputtering, reactive sputtering, atomic layer deposition (ALD), low pressure chemical vapor deposition (LPCVD), plasma enhanced chemical vapor deposition (PECVD), metal organic chemical vapor deposition (MOCVD), etc. Although electrodes 333 and 335 are illustrated as fully covering the bottom surface of the well, the electrodes can be formed to only partially cover the bottom of the well; that is, not extend from one sidewall to the other or to partially lie adjacent to some of the sidewalls but not to others. In some embodiments, the electrodes can be formed to partially extend up the sidewall of the well. In some embodiments, the electrodes (e.g., the pattern electrodes or the common electrode) can be formed of conductive materials, such as metals, semi-metals, conductive ceramics, other conductive materials, or a combination thereof or any other suitable conductive material. Exemplary metals include transition metals or non-transition metals. For example, the transition metals may include tungsten, titanium tantalum, hafnium, zirconium, gold, silver, platinum, copper, alloys thereof, or any combination thereof. A non-transition metal may include aluminum. The metal may be a noble metal such as gold, silver, platinum, alloys thereof, or combinations thereof. Other conductive materials can include graphene, conductive polymers, cermets, ceramics, or doped semiconductors. Conductive ceramics may includes a nitride, such as a titanium nitride. Semiconductors may include doped silicon, doped gallium arsenide, indium tin oxide, or combinations thereof. During manufacturing and/or operation of the device, a thin oxide of the material of the conductive material may grow/be grown at the surface of the electrode(s). The presence of an oxide depends on the conductive material, the manufacturing processes performed, and the conditions under which the device is operated. In some embodiments, a conductive material may be titanium nitride, and titanium oxide or titanium oxynitride may be grown on the inner surface of the conductive material (or on the patterned electrode) during manufacturing and/or during exposure to solutions during use. In some embodiments, the conductive material may have a volume resistivity of not greater than 6.0×10⁷ ohm-m at 25° C. In some embodiments, the volume resistivity may be not greater than 1.0×10⁷ ohm-m at 25° C., such as not greater than 5.0×10⁶ ohm-m at 25° C., or not greater than 2.0×10⁶ ohm-m at 25° C., but generally greater than 10-9 ohm-m at 25° C. In a particular example, the electrode surface can be separated from a solution by an insulator material or a semiconductor material. In some embodiments, the insulator can be a metal oxide, a semi-metal oxide, or an oxynitride of a metal or semi-metal. The insulator may include an oxide of silicon, titanium, zirconium, hafnium aluminum, tantalum, or a combination thereof. Additionally, the insulator may include a titanium oxynitride or silicon oxynitride.

The solid support may be of varied size, as would be understood by one of ordinary skill in the art. Ideally, each well may have one solid support therein. The solid supports may either be the same size or a different size as other solid supports in other wells of the array. The size of the solid support may be chosen based on well size and vice versa, or the solid support size may be made independent of well size and vice versa. In some embodiments, the well depth may be approximately equal to the diameter of the solid support. In such an embodiment, an electrode on the surface of the well and the bottom of the well would be within the Debye distance. In some embodiments, deeper wells may be used. In some embodiment, common electrodes may be annular rings inside the well. In some embodiments, the common electrode of each well is a common electrode to provide the advantage of having one electrode common across all sensor wells on a chip. Thickness of metallization in a buried layer can be limited, and with current for all wells flowing through the common electrode, this embodiment may result in large well-to-well variation. In some embodiment, the top surface metallization can serve as the common electrode, providing thicker metal deposition and lower resistance. In such an arrangement, multiple bond wires at multiple locations would connect this top surface metal to the reference potential source.

The use of AC excitation may provide a benefit of allowing narrowband filtering of the measured signal. This may provide a large reduction in noise. Alternatively, synchronous rectification may be employed. This can provide high discrimination of the desired signal from noise or interfering sources. In some embodiments, conductance may be measured by applying a constant alternating current (AC) voltage across the electrodes, and then measuring the resulting current. Obtaining accurate, high-value resistance may be difficult in integrated circuits. Accordingly, in some embodiments, a current/voltage converter circuit may be provided. Therefore, current excitation may be preferred for an integrated circuit implementation. Current sources may be more easily implemented in semiconductor technology, and large numbers of identical current sources may be provided using only one transistor per source. The voltage appearing across a current source may be measured directly or amplified. In some embodiments, the bead's double-layer interface with an electrolyte fluid can have a complex impedance, such as, for example, capacitance in addition to conductance. The sensor plate interfaces of each well to the fluid can look capacitive; thus, the use of AC excitation may provide another dimension of measurement, by measuring at different frequencies, e.g. electrochemical impedance spectroscopy (EIS). This may be performed on a semiconductor chip, using synchronous rectification, multiplying the measured signal with two orthogonal phases of the source frequency, averaging the results, and thereby getting two values (real and imaginary components of the impedance) at each measured frequency and well. This can provide measurement of the complex frequency response while providing high noise rejection. Assuming the low pass filter averages 100 s of cycles of the AC signal, noise reduction can exceed 20 dB. Alternatives include simple full-wave detection of the measured quantity, or combining either synchronous detection or full-wave detection with pre-filters.

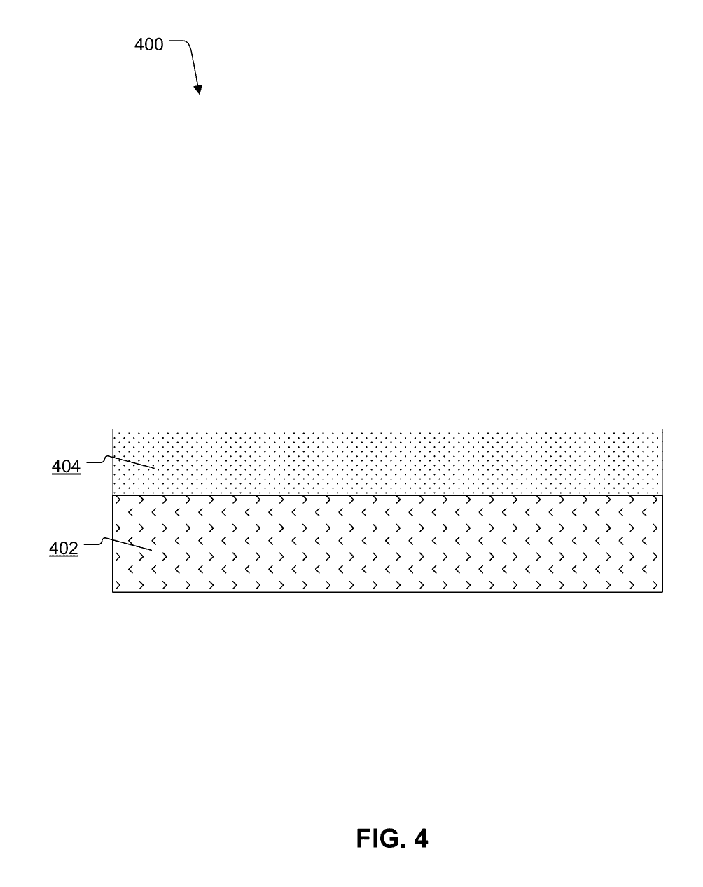

FIGS. 4-11 illustrate stages in a manufacturing process for forming an array of sensors and corresponding well structures according to an exemplary embodiment. FIG. 4 illustrates a structure 400 including dielectric layer 404 deposited on substrate 402 Dielectric layer 404 can comprise any suitable dielectric/insulator and can be deposited using techniques known to those of ordinary skill in the art. In some embodiments, the thickness of dielectric layer 404 may be 2 kÅ although any suitable thickness may be used.

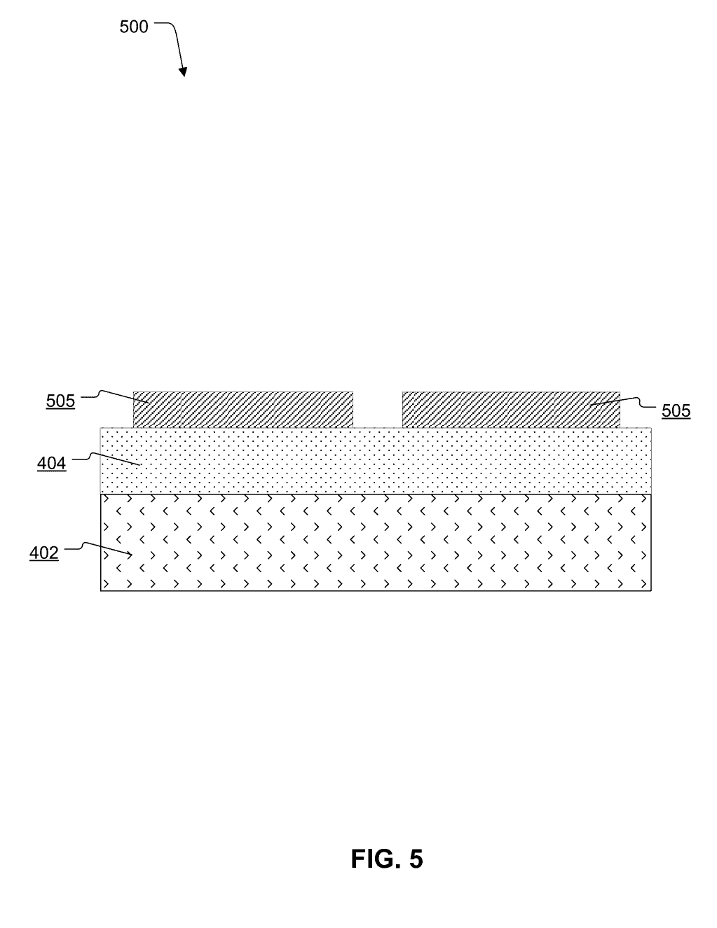

As illustrated in FIG. 5, patterned electrodes 505 can be formed on the dielectric layer 404 of structure 500 using techniques known in the semiconductor industry. In some embodiments, the thickness of the patterned electrodes at this stage can be 2 kÅ, for example. In some embodiments, the electrodes (e.g., the pattern electrodes or the common electrode) may be formed of conductive materials, such as metals, semi-metals, conductive ceramics, other conductive materials, or a combination thereof, or any other suitable conductive material. The metals may include transition metals or non-transition metals. The transition metals may include tungsten, titanium tantalum, hafnium, zirconium, gold, silver, platinum, copper, alloys thereof, or any combination thereof. A non-transition metal may include aluminum. In some embodiments, the metal may be a noble metal such as gold, silver, platinum, alloys thereof, or combinations thereof. Other conductive materials may include graphene, conductive polymers, cermets, ceramics, or doped semiconductors. A ceramic may include a nitride, such as a titanium nitride. Exemplary semiconductors may include doped silicon, doped gallium arsenide, indium tin oxide, or combinations thereof During manufacturing and/or operation of the device, a thin oxide of the material of the conductive material may grow/be grown at the surface of the electrode(s). The presence of an oxide depends on the conductive material, the manufacturing processes performed, and the conditions under which the device is operated. In some embodiments, a conductive material may be titanium nitride, and titanium oxide or titanium oxynitride may be grown on the inner surface of the conductive material (or on the patterned electrode) during manufacturing and/or during exposure to solutions during use. In some embodiments, the conductive material may have a volume resistivity of not greater than 6.0×10⁷ ohm-m at 25° C. In some embodiments, the volume resistivity may be not greater than 1.0×10⁷ ohm-m at 25° C., such as not greater than 5.0×10⁶ ohm-m at 25° C., or not greater than 2.0×10⁶ ohm-m at 25° C., but generally greater than 10-9 ohm-m at 25° C. In a particular example, the electrode surface can be separated from a solution by an insulator material or a semiconductor material. In some embodiments, the insulator can be a metal oxide, a semi-metal oxide, or an oxynitride of a metal or semi-metal. The insulator may include an oxide of silicon, titanium, zirconium, hafnium aluminum, tantalum, or a combination thereof. Additionally, the insulator may include a titanium oxynitride or silicon oxynitride.

Electrode 505 is illustrated as having been formed directly on dielectric 404; however, additional layers can optionally be formed between electrode 505 and substrate 404 or between electrode 505 and substrate 402. For example, routing layers for accessing each electrode of the patterned electrodes in the array may be formed in layers beneath the electrode. Alternatively, each electrode of the patterned electrodes in the array may be accessed directly.

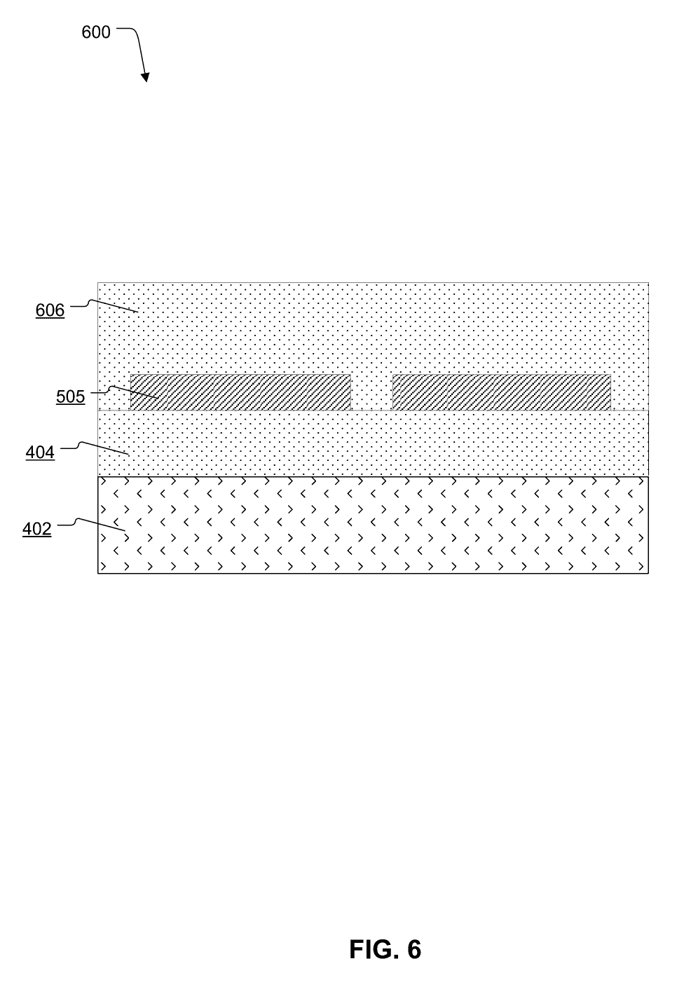

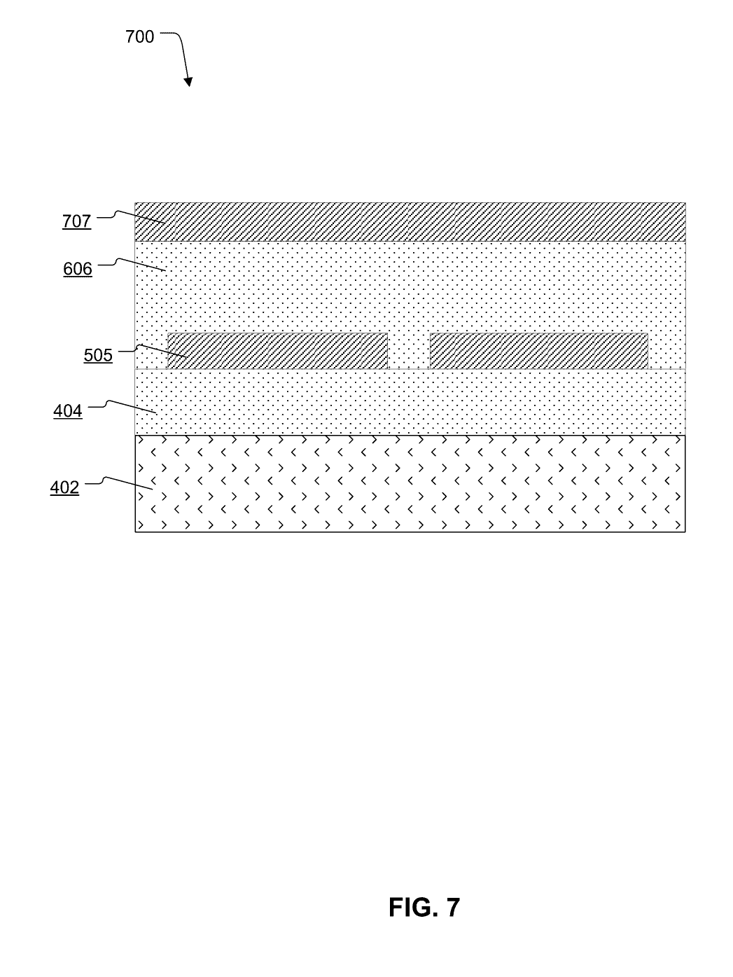

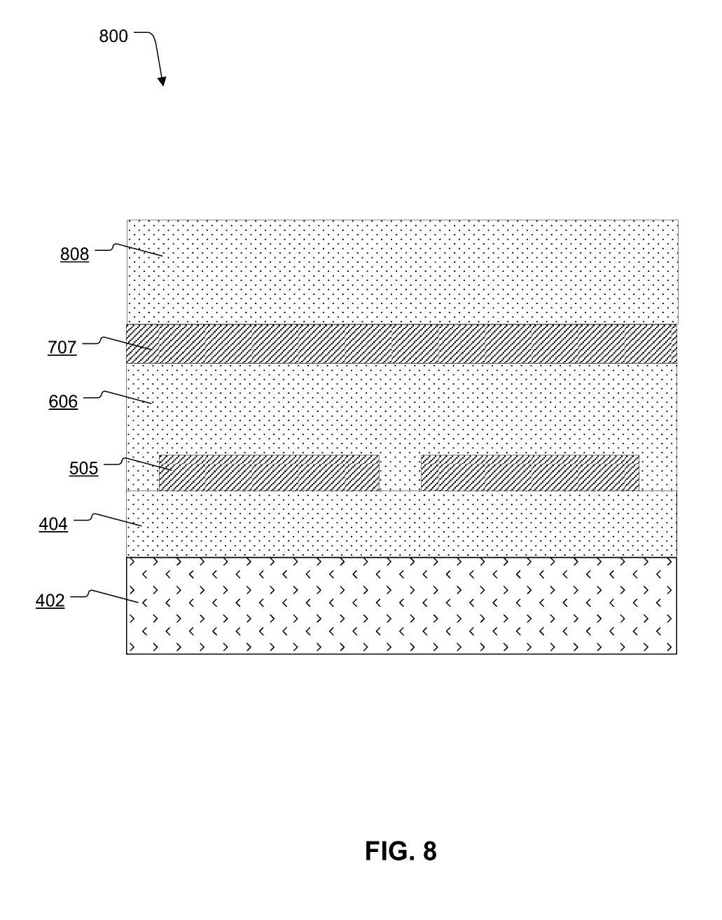

As shown in FIG. 6, dielectric layer 606 is deposited over patterned electrodes 505 resulting in structure 600. In some embodiments, the thickness of dielectric layer 606 at this stage may be 14 kÅ or any other suitable thickness. Conductive material 707 is then deposited over dielectric layer 606 resulting in structure 700 illustrated in FIG. 7. Conductive material 707 may be any of the conductive materials discussed above. The conductive material 707 can be the same as or can be different from the material selected to form the patterned electrodes 505. In some embodiments, the thickness of conductive material 707 at this stage can be 2 kÅ or any other suitable thickness. The thickness of conductive material 707 can be the same or different as the thickness of the patterned electrodes 505. Shapes including but not limited to sawtooth and triangular, for example, may be used for the shape of the conductive material to increase area of interaction between the conductive material and the bead/analyte/queried area or volume (e.g. microwell). Fluids may flow across the chip or there can be one port of entry and exit. Next, a dielectric layer 808 is deposited over conductive material 707 of structure 700 in FIG. 7, resulting in structure 800 as illustrated in FIG. 8. In some embodiments, the thickness of dielectric layer 808 at this stage can be 14 kÅ or any suitable thickness.

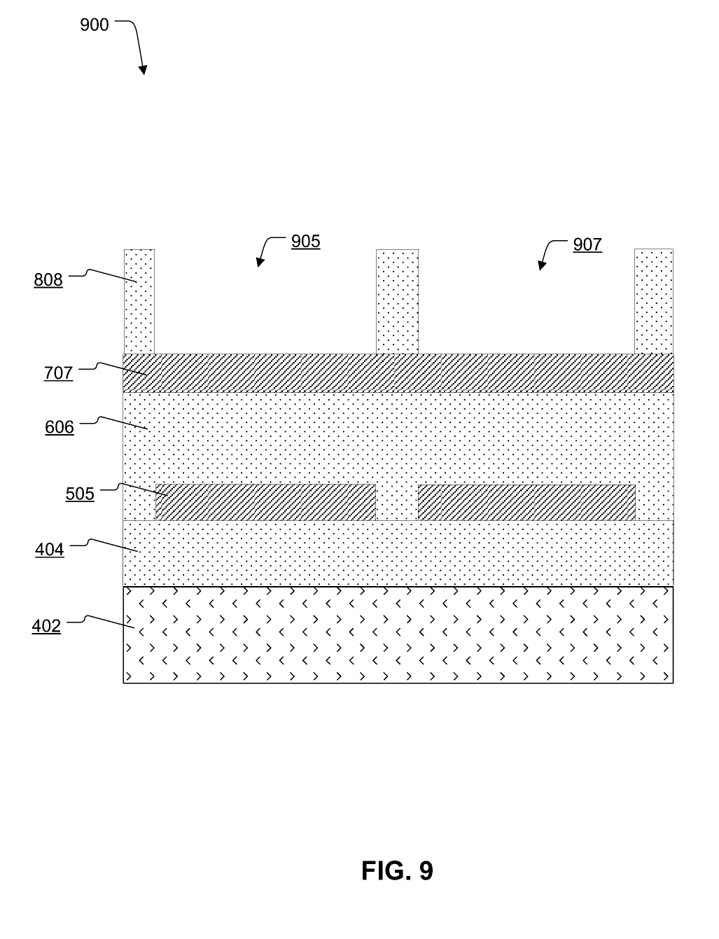

As shown in FIG. 9, openings 905, 907 can be formed by using a lithographic process. The lithographic process may involve patterning a layer of photoresist on the dielectric material 808 to define the locations of the openings 905, 907, and then anisotropically etching the dielectric material using the patterned photoreist as an etch mask. The resulting structure 900 is shown in FIG. 9. The anisotropic etching of the dielectric material can, for example, be a dry etch process, such as a fluorine based Reactive Ion Etching (RIE) process.

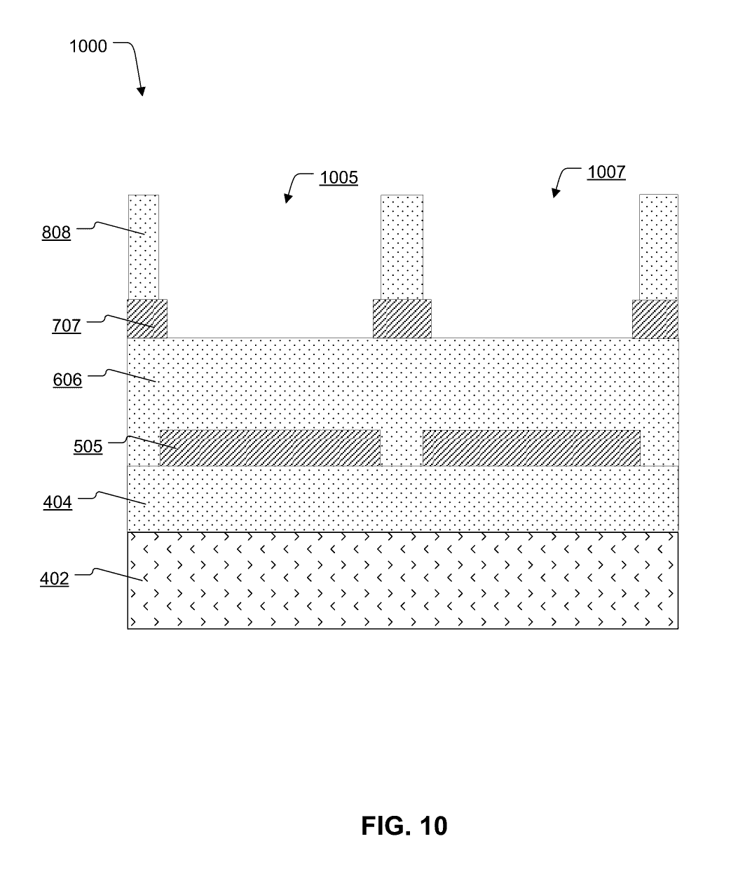

Next, openings 1005, 1007 can be formed by using a lithographic process, for example, to pattern a layer of photoresist on conductive material 707 to define the locations of the openings 1005, 1007, and then anisotropically etching the conductive material 707 using the patterned photoreist as an etch mask resulting in structure 1000 as illustrated in FIG. 10. The anisotropic etching of the conductive material can, for example, be a dry etch process, such as a fluorine based Reactive Ion Etching (RIE) process.

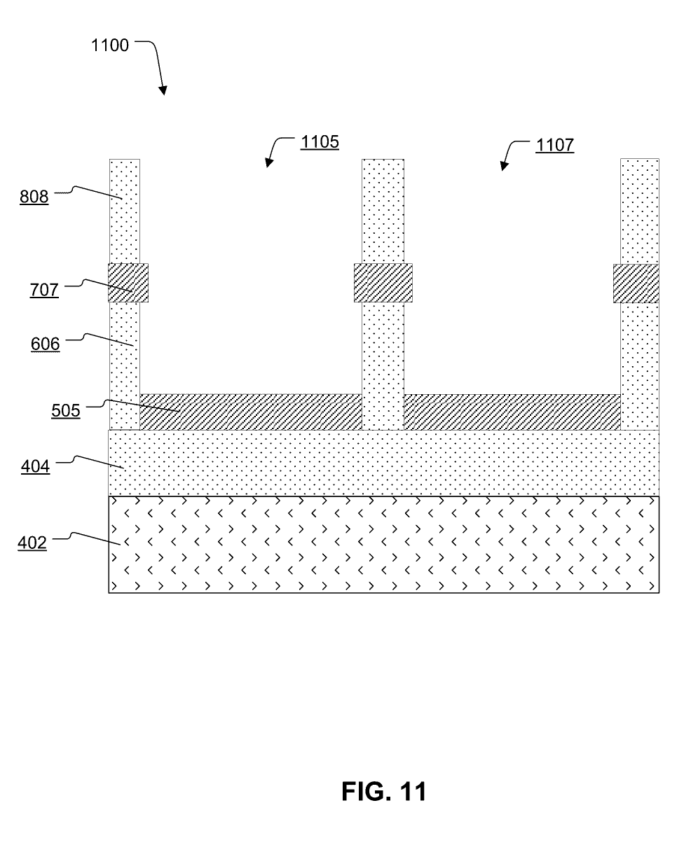

Openings 1105, 1107 can, for example, be formed by using a lithographic process to pattern a layer of photoresist on dielectric material 606 to define the locations of the openings 1105, 1107, and then anisotropically etching dielectric material 606 using the patterned photoreist as an etch mask resulting in structure 1100 illustrated in FIG. 11. The anisotropic etching of the dielectric material can, for example, be a dry etch process, such as a fluorine based Reactive Ion Etching (RIE) process. In some embodiments, the etching of the conductive material can result in the conductive material being flush with the sidewall of the well. In some embodiments, as illustrated in FIG. 11, etching of conductive material 707 can result in the conductive material protruding into the opening formed by the etching of the dielectric on both sides of the conductive material. In some embodiments, the opening may be formed by etching the conductive element and dielectric materials in one step. The location of the conductive material may be fabricated to be anywhere within the dielectric.

Although only one layer of conductive material is illustrated, more than one layer of conductive material may be deposited within the wall of the microwell such that the well wall comprises alternating dielectric material and conductive material (e.g. number of layers of conductive material deposited within the wall of the microwell can be greater than one). The thickness of the conductive material can vary as one of ordinary skill in the art would recognize. Appropriate etching would follow the order of materials used to create the well. For example, if the conductive material is at the top of the well and there is only one dielectric layer therebeneath, etching would be a two-step process; first etching the conductive material and then the dielectric material. For a microwell having a conductive material at bottom top of the well and only one dielectric layer thereabove, etching would be a two-step process, first etching the dielectric material and then the conductive material. In such a case, the conductive material would be separated from the patterned electrodes by an insulator. Optionally, metal may be electroplated onto the conductive material once exposed after the etching steps. For example, the exposed surface of conductive material 707 may be coated with a thin layer of platinum, or another material suitable for electroplating.

Although patterned electrode 505 is illustrated as fully covering the bottom surface of the well, the patterned electrodes may be formed to only partially cover the bottom of the well; that is, not extend from one sidewall to the other. Additionally, the patterned electrodes can be formed to partially extend up the sidewall of the well. For example, the patterned electrodes can extend at least 5% up the sidewall of the well, such as at least 10%, at least 20%, at least 30%, at least 40%, at least 50%, at least 60%, at least 70%, at least 80%, at least 90%, or even at least 95% up the sidewall of the well. The upper surface 10 of the well wall structure may be free of conductive material 707 or conductive material 707 may overhang at least a portion of the well wall structure. In some embodiments, the patterned electrodes may protrude into the wells.

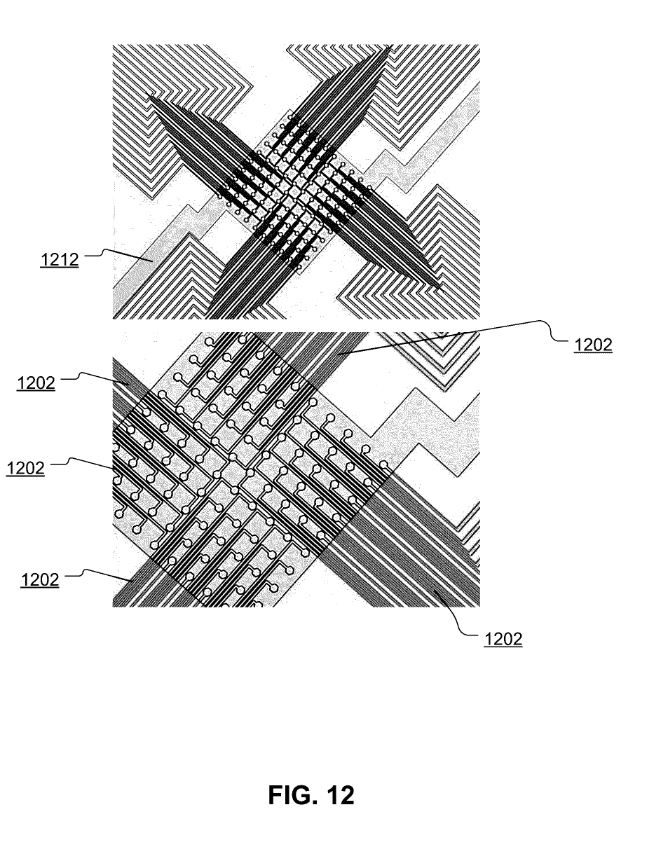

FIG. 12 illustrates an exemplary routing scheme of the integrated circuit. For exemplary purposes only, the patterned electrodes can be individually addressable and connections can be made such that an electrode at a bottom of a well is read out on a dedicated readout line as shown in FIG. 12. The conductive material as described above with reference to FIGS. 7-11 (conductive material 707) may be a global electrode (e.g. 1212) in a dedicated layer different from the layer the patterned electrodes (e.g. 1202) are formed within. The global electrode can be common to all or some of the wells of the array.

In a particular example, the system and devices may be used to analyze the nature of biomolecules, such as nucleic acids or proteins. For example, copies of a molecule may be deposited into a well, and changes in the dielectric or electrical characteristics in response to specific changes in the molecule may be used to determine characteristics of the molecules. For example, the dielectric or electrical characteristic detected may include a change in the impedance, capacitance, inductance, conductance or resistance, or a change in resonant frequency.

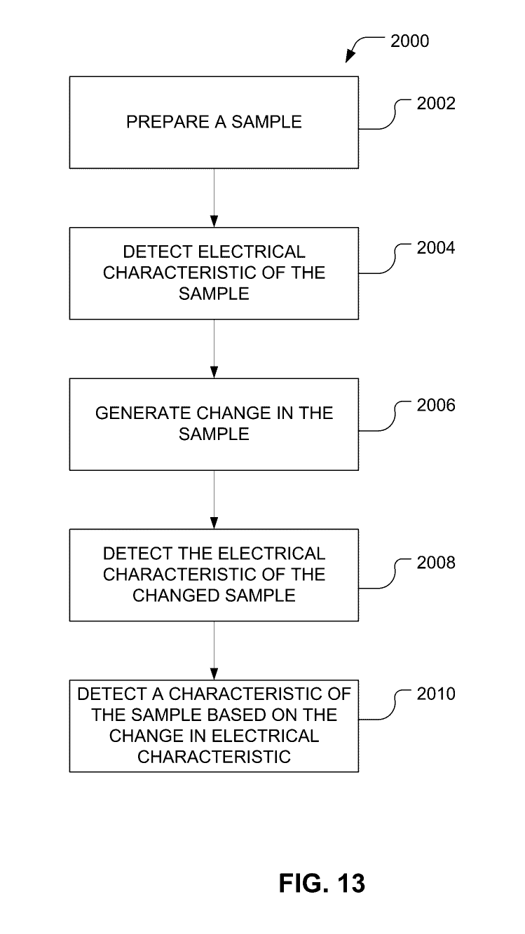

In an example illustrated in FIG. 13, a method 2000 includes preparing a sample, as illustrated at 2002. In some embodiments, preparing the sample may include depositing copies of the biomolecule into a well. For example, solid supports (such as hydrogel particles) including a monoclonal population of molecules may be deposited into the well. In some embodiments, the well may include a conformal hydrogel network onto which a monoclonal population of the molecule is generated. In some embodiement, a molecule may be and attached to surface agents within the well. In some embodiments, a molecule can be a nucleic acid which is amplified using polymerase chain reaction (PCR), recombinase polymerase amplification (RPA), rolling circle amplification, other amplification techniques, or any combination thereof. Additionally, a primer and an enzyme or polymerase can be applied to the nucleic acid to facilitate nucleotide or probe incorporation or chain extension. An electrical characteristic of the sample 2004 may be detected. For example, the electrical characteristic may be impedance. The impedance may be measured in a chemical system that lacks a redox reaction. Alternatively, the system may be designed to incorporate a redox reaction.

In a particular example, impedance may be measured using a frequency signal generated across the electrodes. The frequency signal may be a single frequency. Alternatively, the impedance may be measured using multiple frequencies. In some embodiments, the impedance may measured using a complex waveform. Two or more frequencies or patterns may be added or applied concurrently. Alternatively, two or more frequencies or patterns can be applied consecutively or patterns may include portions that are concurrent and consecutive. In some embodiments, the frequencies may be selected from frequencies in a range of 10 Hz to 1 MHz, 70 Hz to 1 MHz, 100 Hz to 500 kHz, or 100 Hz to 10 kHz. The pattern may include a sinusoidal pattern, square pattern, saw tooth pattern, or a combination thereof.

As illustrated at 2006 of FIG. 13, a change may be generated in a sample. In some embodiments, the molecular size of the biomolecule or a charge of the biomolecule may be manipulated. Specific probes may be added to the biomolecule or the biomolecule may be cleaved. Where the biomolecules includes a nucleic acid or protein, the molecular size may be increased by polymerization, for example, by nucleotide addition to DNA or RNA or protein synthesis. In a particular example, the size of a biomolecule may be increased, for example, by extension of a primer and incorporation of a nucleotide or using a ligation probe. In particular, one of a set of nucleotides may be applied through flow cell of the system and incorporated along the nucleic acid depending on the sequence of the nucleic acid. Optionally, the nucleic acid probe, nucleotide or primer may utilize the ribose or deoxyribose nucleotides, protein analogs or other analogs thereof, or a combination thereof.

In some embodiments, the molecular size of the biomolecules may be decreased. For example, the molecular size may be decreased by sequential or non-sequential digestion, for example, by exonuclease digestion of a nucleic acid or by protease digestion of protein.

In a further example, the molecular size may be altered by the association of additional molecules, such as binding probes or moieties, to the biomolecules. For example, the molecular size may be manipulated by applying a moiety to an existing molecule, for example, by hybridization of an oligonucleotide to DNA or RNA or of an antibody or antigen to the biomolecule.

In an additional example, the dissociation of additional molecules may be used to alter the molecular size of the biomolecules, for example, the dissociation or release of hybridize or bound probes.

As illustrated in FIG. 13, the electrical characteristic can be tested to determine a change in the electrical characteristic in response to a change sample 2008. The electrical characteristic may be detected, such as detecting impedance using frequencies as have been described previously.

In some, the detection of the electrical characteristic may take place in low ionic strength solutions. For example, the ionic strength of the solution may be equivalent to a saline solution having a concentration of 10 μM to 1 mM, such as 10 μM to 100 μM, 10 μM to 90 μM, or 10 μM to 70 μM.

The characteristic of the samples, such as a characteristic of biomolecules, may be detected based on the change in the electrical characteristic, as illustrated in FIG. 13 at 2010. For example, a change in impedance in response to the incorporation of a nucleotide may be used to detect the sequence of a nucleic acid. In another example, the association or dissociation of an oligonucleotide probe to a nucleic acid sample may be detected based on a change in impedance and may indicate the presence or absence of a specific sequence within the nucleic acid sample.

Note that not all of the activities described above in the general description or the examples are required, that a portion of a specific activity may not be required, and that one or more further activities may be performed in addition to those described. Still further, the order in which activities are listed are not necessarily the order in which they are performed.

In the foregoing specification, the concepts have been described with reference to specific embodiments. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of invention.

As used herein, the terms “comprises,” “comprising,” “includes,” “including,” “has,” “having” or any other variation thereof, are intended to cover a non-exclusive inclusion. For example, a process, method, article, or apparatus that comprises a list of features is not necessarily limited only to those features but may include other features not expressly listed or inherent to such process, method, article, or apparatus. Further, unless expressly stated to the contrary, “or” refers to an inclusive-or and not to an exclusive-or. For example, a condition A or B is satisfied by any one of the following: A is true (or present) and B is false (or not present), A is false (or not present) and B is true (or present), and both A and B are true (or present).

Also, the use of “a” or “an” are employed to describe elements and components described herein. This is done merely for convenience and to give a general sense of the scope of the invention. This description should be read to include one or at least one and the singular also includes the plural unless it is obvious that it is meant otherwise.

Benefits, other advantages, and solutions to problems have been described above with regard to specific embodiments. However, the benefits, advantages, solutions to problems, and any feature(s) that may cause any benefit, advantage, or solution to occur or become more pronounced are not to be construed as a critical, required, or essential feature of any or all the claims.

After reading the specification, skilled artisans will appreciate that certain features are, for clarity, described herein in the context of separate embodiments, may also be provided in combination in a single embodiment. Conversely, various features that are, for brevity, described in the context of a single embodiment, may also be provided separately or in any subcombination. Further, references to values stated in ranges include each and every value within that range.

1. A sensor comprising:
a substrate having a first reaction region and a second reaction region;
a first electrode associated with the first reaction region;
a second electrode associated with the second reaction region; and
a third electrode wherein the third electrode is common to both the first reaction region and the second reaction region.

2. The sensor of claim 1 wherein the third electrode is located within a debye length of the first electrode.

3. The sensor of claim 2 wherein the third electrode is located within a debye length of the second electrode.

4. The sensor of claim 1 further including an oxide layer on the first electrode.

5. The sensor of claim 1 further including biocompatible material on the surface of the first electrode and the second electrode.

6. A method for detecting a biological reaction comprising:
providing a reaction region including a nucleic acid sample;
detecting a first impedance level of the reaction region;
exposing the reaction region to a reagent solution including a targeting biological molecule;
detecting a second impedance level of the reaction region;
comparing the first impedance level and the second impedance level; and
determining whether a change in impedance has occurred.

7. The method of claim 6 wherein the change in impedance is approximately zero.

8. The method of claims 6 wherein the change in impedance is an increase.

9. The method of claim 6 further including determining the sequence of the nucleic acid sequence.

10. The method of claim 6 wherein the determining indicates an incorporation event.

11. A method of manufacturing a sensor comprising
providing a substrate;
depositing a metal layer the substrate in a predetermined location;
depositing a first dielectric material layer on the metal layer;
depositing a conductive layer on the first dielectric material layer;
depositing a second dielectric material layer on the conductive layer; and
etching the second dielectric material layer, the conductive layer, and the first dielectric layer,
wherein the second dielectric material layer, the conductive layer, and the first dielectric layer are etched in one step,
wherein the etching exposes the metal layer to the reaction region.

12. The method of claim 11 wherein the etching includes etching the second dielectric layer thereby exposing the conductive layer to the reaction region, etching the conductive layer thereby exposing the first dielectric layer to the reaction region, and etching the first dielectric layer thereby exposing the metal layer to the reaction region.

13. The method of claim 11 wherein depositing the first dielectric material includes depositing an amount of dielectric material such that the depositing of a conductive layer occurs at a debye length from the metal layer.
